(19) United States
(12) Patent Application Publication
(10) Pub. No.: US 2025/0288285 A1
(43) Pub. Date: Sep. 18, 2025

(54) FUNCTIONALIZED NANOMATERIAL FOR MEDICAL ANALYTE COLLECTION

(71) Applicant: Cellect Laboratories Inc., Windsor (CA)

(72) Inventors: Claire Theresa Murphy, Kitchener (CA); Brandon William Klassen, Windsor (CA); Minren Hung, Windsor (CA); Lauren Sarah Prophet, Windsor (CA)

(21) Appl. No.: 19/070,246

(22) Filed: Mar. 4, 2025

Related U.S. Application Data
(60) Provisional application No. 63/566,776, filed on Mar. 18, 2024.

(51) Int. Cl. A61B 10/02; A61B 5/00; A61B 5/15; A61F 13/20; A61F 13/47; A61F 13/472; A61F 13/512

(52) U.S. Cl. CPC A61B 10/0291; A61B 5/150045; A61B 5/150358; A61B 5/4331; A61F 13/202; A61F 13/2071; A61F 13/472; A61F 13/512; A61F 2013/4708

(57) ABSTRACT

A device for collection of vaginal discharge or menstrual blood as an analyte of medical testing, comprising a filter configured to separate cells of interest from other material and a nanomaterial cartridge comprising a nanomaterial configured to passively adsorb DNA and segregate cells of different sizes. The nanomaterial is a silica sol gel with templated macropores

### CROSS-REFERENCE TO RELATED APPLICATION(S)

This application claims the benefit under 35 U.S.C. § 119(e) to U.S. Provisional Application 63/566,776, filed Mar. 18, 2024, and entitled Nanomaterial Functionalized Menstrual Products for Medical Diagnostic Analyte Collection, which is hereby incorporated herein by reference in its entirety for all purposes.

### TECHNICAL FIELD

The disclosure relates to medical diagnostic testing, and more specifically to medical diagnostic testing utilizing a nanomaterial for DNA and RNA targeting.

### BACKGROUND

Cervical cancer is a very common cancer in women around the world. Cervical cancer screening currently is conducted by visual inspection with acetic acid, HPV DNA testing, and/or Papanicolaou testing (Pap smear). These tests currently require clinic based cell sampling techniques. These cell sampling techniques can include cervical visualization using a speculum, sample collection using a brush or speculum, and fixation on a glass slide or suspension in liquid. Because these known tests can be invasive and uncomfortable there is a high rate of non-compliance for recommended regular screening.

Nanomaterials describe materials of which a single unit is sized in at least one dimension between 1 and 100 nm. Nanomaterial research takes a materials science-based approach to nanotechnology, leveraging advances in materials metrology and synthesis which have been developed in support of microfabrication research. Materials with structure at the nanoscale often have unique optical, electronic, thermophysical or mechanical properties.

A medical test is a medical procedure performed to detect, diagnose, or monitor diseases, disease processes, susceptibility, or to determine a course of treatment. Medical tests such as physical and visual exams, diagnostic imaging, genetic testing, chemical and cellular analysis, relating to clinical chemistry and molecular diagnostics, are typically performed in a medical setting. A diagnostic test is a procedure performed to confirm, determine, or otherwise aid in the diagnosis or detection of disease in an individual suspected of having a disease, usually following the report of symptoms, or based on other medical test results. Diagnostic tests can also be used to provide prognostic information on people with established diseases.

### BRIEF SUMMARY

In Example 1, a device for collection of vaginal discharge for medical testing comprising a filter and a nanomaterial in fluidic communication with the filter, wherein the filter is configured to

Example 2 relates to the device of any of Examples 1 and 3-9, wherein the vaginal discharge comprises menstrual blood.

Example 3 relates to the device of any of Examples 1-2 and 4-9, further comprising an absorbent layer in fluidic communication with the nanomaterial.

Example 4 relates to the device of any of Examples 1-3 and 5-9, wherein the device is a nanomaterial embedded tampon.

Example 5 relates to the device of any of Examples 1-4 and 6-9, wherein the device is a nanomaterial embedded pad comprising at least a sticker layer.

Example 6 relates to the device of any of Examples 1-5 and 7-9, wherein the device is a nanomaterial embedded underwear comprising at least a fabric upper.

Example 7 relates to the device of any of Examples 1-6 and 8-9, wherein the device is a nanomaterial embedded sticker configured for placement in an undergarment.

Example 8 relates to the device of any of Examples 1-7 and 9, wherein the nanomaterial is a silica sol gel with templated macropores.

Example 9 relates to the device of any of Examples 1-8, wherein the filter is configured to trap cells of interest and allow other material to pass through while the nanomaterial is configured to bind DNA.

In Example 10, a device for collection of vaginal discharge or menstrual blood as an analyte of medical testing, comprising a filter configured to separate cells of interest from other material and a nanomaterial cartridge comprising a nanomaterial configured to passively adsorb DNA and segregate cells of different sizes.

Example 11 relates to the device of any of Examples 10 and 12-17, wherein the cells of interest are cervical cells.

Example 12 relates to the device of any of Examples 10-11 and 13-17, wherein the medical testing is HPV testing for cervical cancer diagnosis.

Example 13 relates to the device of any of Examples 10-12 and 14-17, wherein the device is selected from a tampon, a pad, an underwear, and a sticker.

Example 14 relates to the device of any of Examples 10-13 and 15-17, further comprising an absorbent layer.

Example 15 relates to the device of any of Examples 10-14 and 16-17, wherein the nanomaterial is a silica sol gel with templated macropores.

Example 16 relates to the device of any of Examples 10-15 and 17, further comprising a permeable layer.

Example 17 relates to the device of any of Examples 10-16, wherein the nanomaterial cartridge may be removed from the device for medical testing.

In Example 18, a nanomaterial embedded sticker for collection of an analyte for medical testing, comprising a mesh layer, a nanomaterial layer adjacent to the mesh layer, a minimum fill area within the nanomaterial configured to indicate a minimum fill for collection, and an adhesive portion at least encircling the nanomaterial layer.

Example 19 relates to the nanomaterial embedded sticker of any of Examples 18 and 20, wherein the mesh layer is configured to trap one or more cells of interest and the nanomaterial layer is configured to adsorb DNA or RNA of interest.

Example 20 relates to the nanomaterial embedded sticker of any of Examples 18 and 19, wherein the nanomaterial is a silica sol gel with templated macropores.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a side view of a filter and nanomaterial of an embedded device, according to one implementation.

FIG. 2 is an expanded view of a tampon implementation of the embedded device, according to one implementation.

FIG. 3 shows the tampon implementation of FIG. 2 in use collecting a sample, according to one implementation.

FIG. 4 is a top view of a pad implementation of the embedded device, according to one implementation.

FIG. 5 is a side, expanded view of a pad implementation of the embedded device, according to one implementation.

FIG. 6 shows the implementation of FIG. 5 in use collecting a sample, according to one implementation.

FIG. 7 is a front, expanded view of an underwear implementation of the embedded device, according to one implementation.

FIG. 8 is a side, expanded view of an underwear implementation of the embedded device, according to one implementation.

FIG. 9 shows the implementation of FIG. 8 in use collecting a sample, according to one implementation.

FIG. 10 is a top view of a sticker implementation of the embedded device, according to one implementation.

FIG. 11 is a side view of a sticker implementation of the embedded device, according to one implementation.

### DETAILED DESCRIPTION

Disclosed herein are various devices, systems, and methods for use of menstrual blood and/or vaginal discharge as an analyte for medical diagnostic tests. In various implementations menstrual blood and/or vaginal discharge are used for human papillomavirus (HPV) testing for cervical cancer diagnosis. The use of the method and system disclosed herein may also be applied to diagnostic testing for other diseases and conditions, as would be appreciated by those of skill in the art. In various implementations, the herein disclosed devices, systems, and methods incorporate a nanomaterial into conventional menstrual products such as tampons, pads, underwear. Various alternative implementations utilize a sticker for collection. Further implementations also include a process for using the nanomaterial to bypass the need for polymerase chain reaction (PCR) in HPV testing.

In various implementations, a sticker, menstrual product (tampon, pad, underwear), or the like, is embedded with a filter, a nanomaterial, and optionally an absorbent layer for the collection of cells, DNA, RNA, and the like for use in medical diagnostic testing, research (such as endometriosis and gynecological research), and the like.

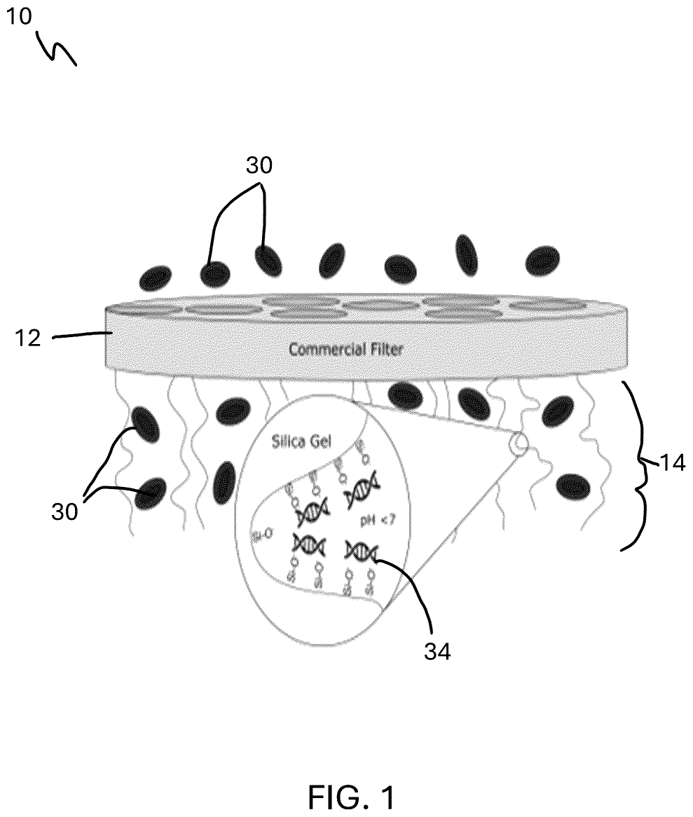

As can be seen in FIG. 1, in certain implementations the embedded device 10 includes a filter 12 and a nanomaterial 14. The filter 12 filters menstrual blood and/or vaginal discharge, passing through various components including DNA 34 and blood cells 30 to the nanomaterial 14 layer. The device 10 may also include an absorbent layer 16, as will be discussed further herein.

In various implementations, the nanomaterial 14 passively adsorbs DNA 34 and RNA while also segregating differently sized cells. A 3D templating synthesis allows for the nanomaterial 14 to form macro pores of various sizes and include surface functionalization. The nanomaterial 14 allows blood cells 30 to pass through the nanomaterial 14 without getting stuck, allowing for passive concentration of target DNA 34 in the nanomaterial 14.

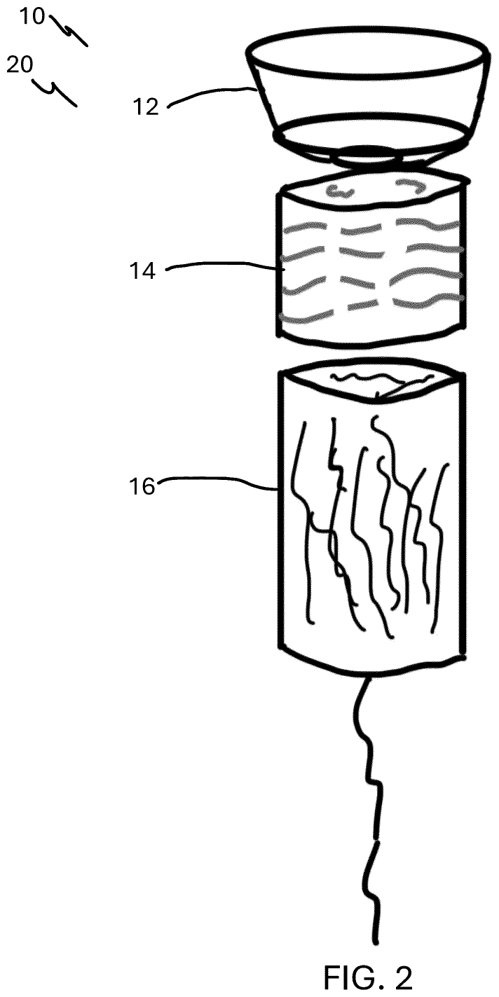

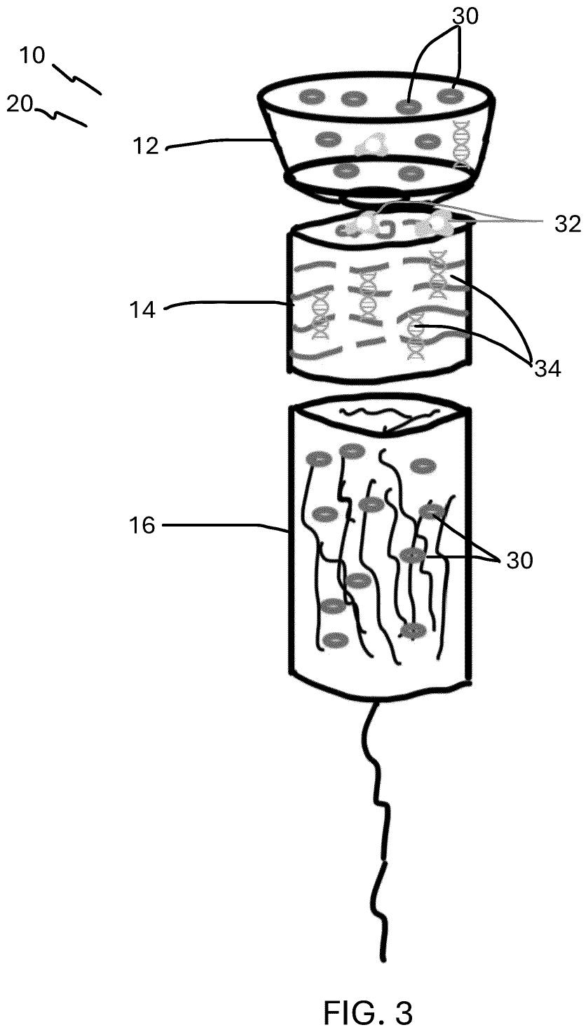

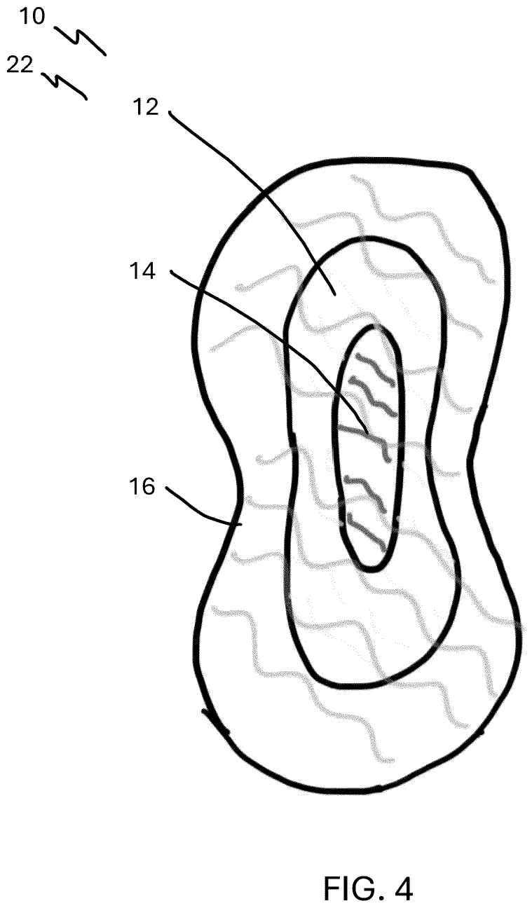

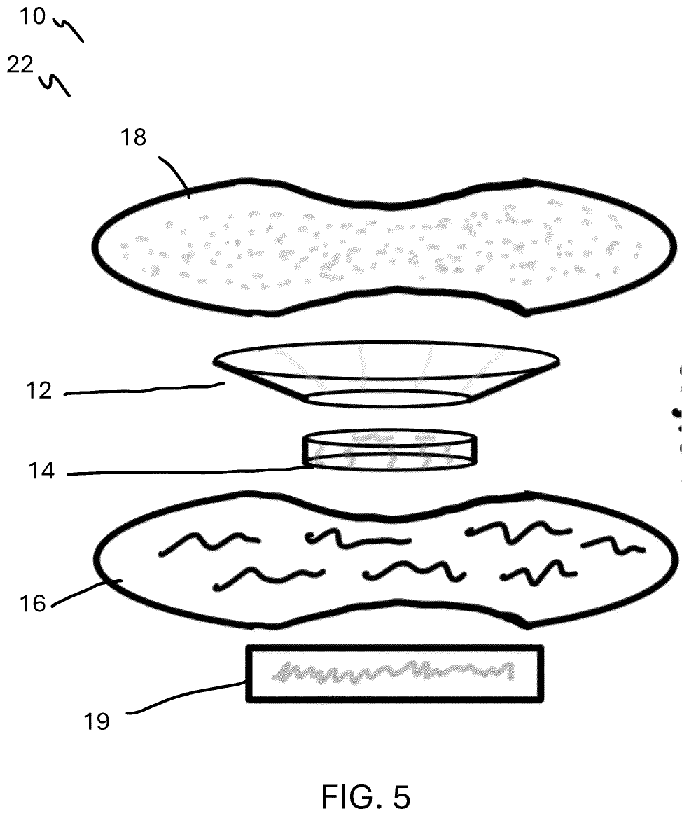

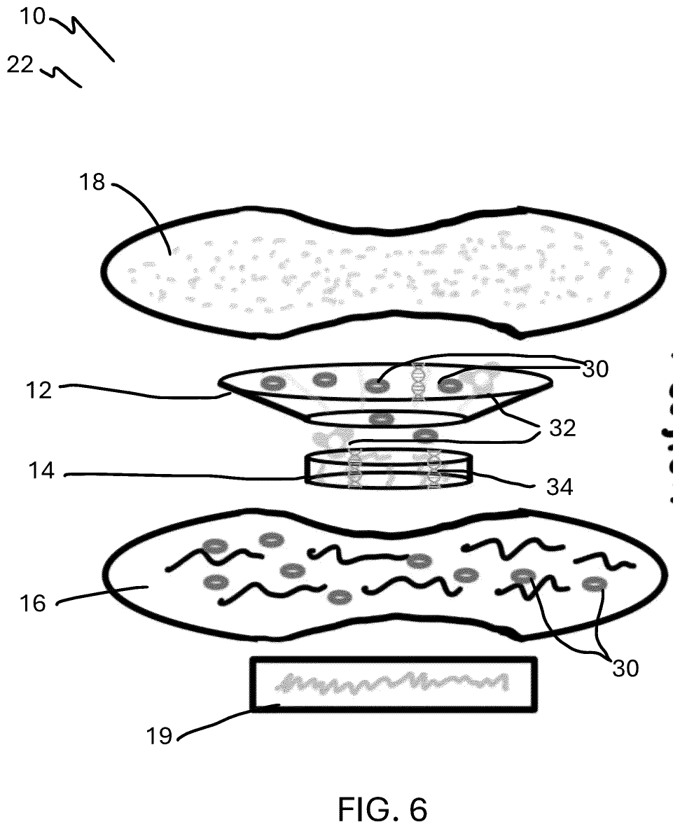

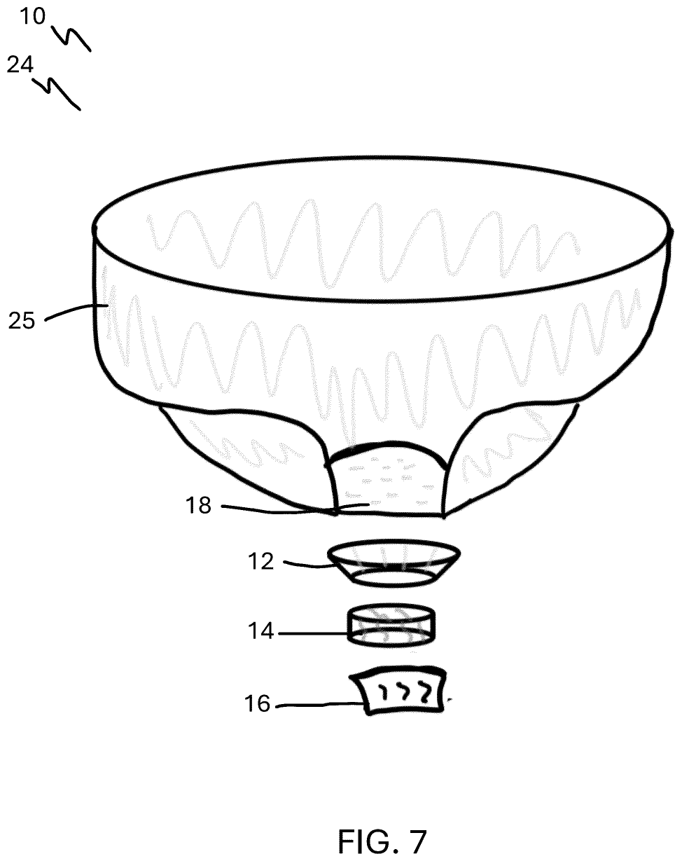

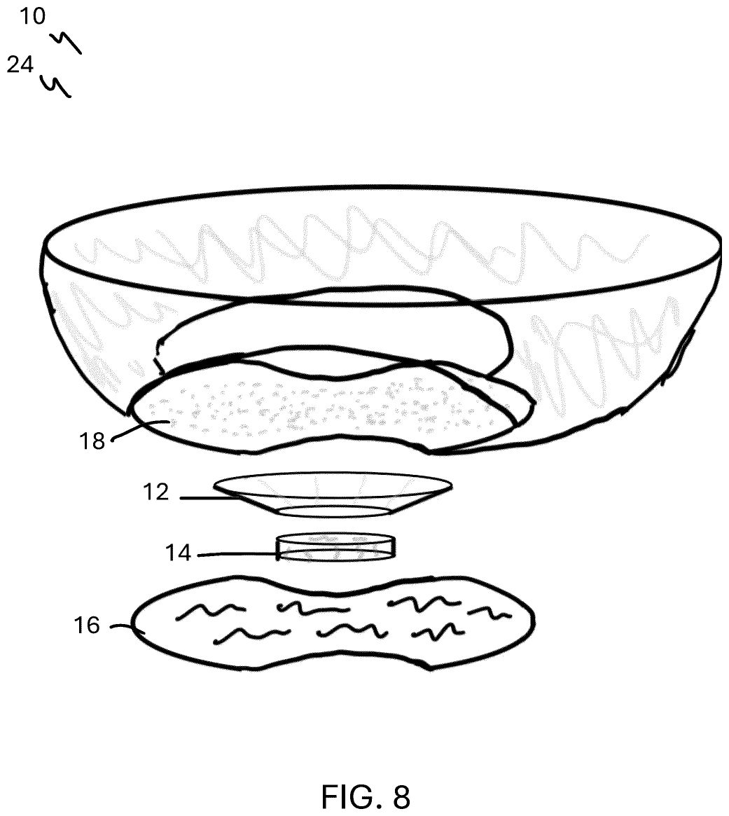

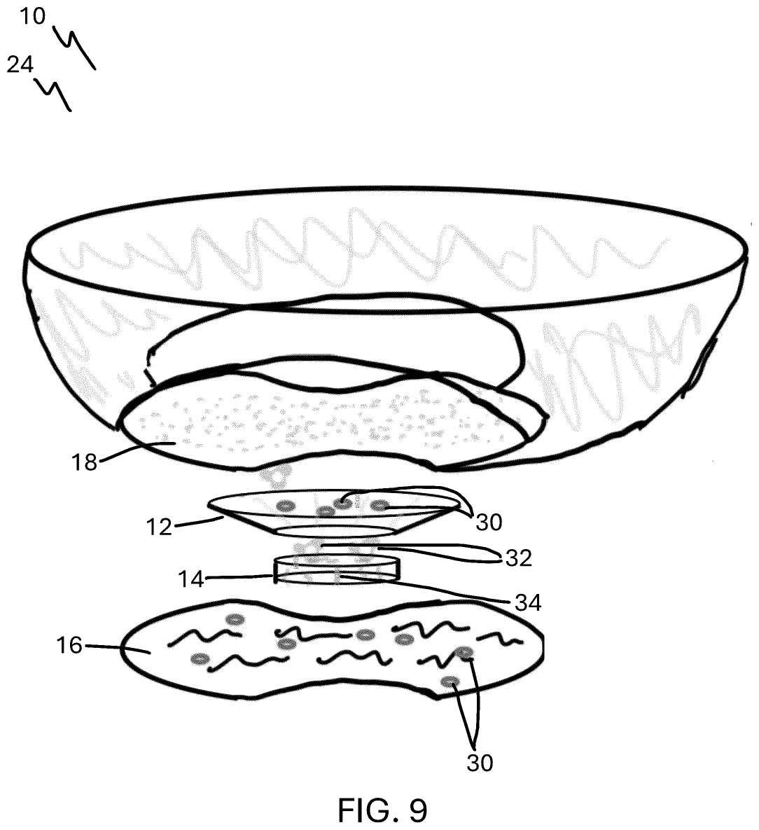

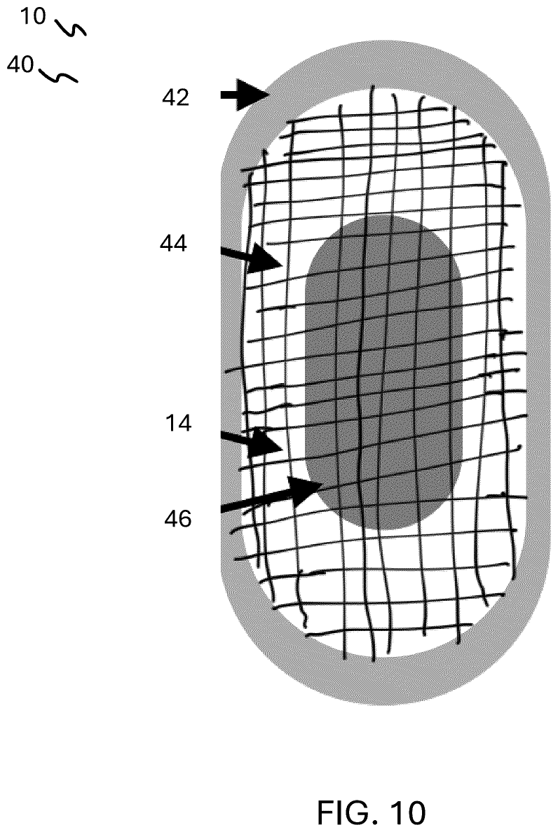

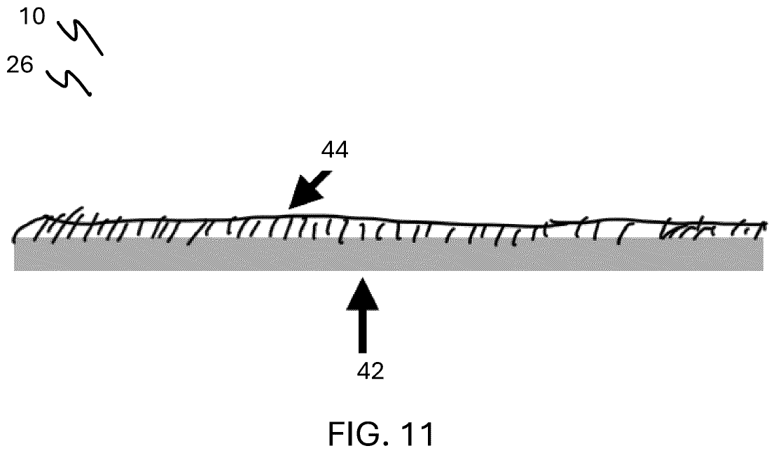

Various implementations of the nanomaterial embedded device 10 are shown variously in FIGS. 2-11 and may include a tampon 20 (FIGS. 2-3), a pad 22 (FIGS. 4-6), underwear 24 (FIGS. 7-9), and the like. Alternative implementations may include a sticker 40 (FIGS. 10-11) configured to be placed in a patient's/user's underwear or alternatively on a conventional menstrual pad for collection. As would be appreciated various modifications to the component parts of the nanomaterial embedded device 10 may differ based on the specific device 10.

In one specific implementation, the nanomaterial embedded device 10 is a modified tampon 10, shown in FIGS. 2-3. These implementations may include a soft material opening including a filter 12, a removable nanomaterial cartridge 14, and an absorbent material layer 16. As would be appreciated, the soft material opening/filter 12 catches the menstrual blood or vaginal discharge, such that the blood/discharge flows down towards the nanomaterial cartridge 14 that catches the cells 32 and nucleic acid 34 of interest while allowing the blood cells 30 to flow and be absorbed by the absorbent layer 16. As will be described further herein, the cartridge 14 may be removed in the lab and the nucleic acid 34 eluted to yield a high concentration sample.

In another specific implementation, the nanomaterial embedded device 10 is a modified pad 22, shown in FIGS. 4-6. These implementations may include a permeable material layer 18, a low friction material layer to include a filter 12, a removable nanomaterial cartridge 14, an absorbent material layer 16, and optionally a sticky underside 19. As would be understood, the permeable layer 18 allows menstrual blood or vaginal discharge to flow to a conical, low friction material layer/filter 12, which feeds into a nanomaterial cartridge 14. The blood cells 30 that are allowed to flow through the cartridge 14 are then absorbed by the absorbent layer 16. The optional sticky underside 19 allows for attachment to undergarments, as would be appreciated. As will be described further herein, the cartridge 14 may be removed in the lab and the nucleic acid eluted to yield a high concentration sample.

In a further specific example, the nanomaterial embedded device 10 may be a modified menstrual underwear 24, shown in FIGS. 7-9. These implementations may be similar to the modified pad implementations of FIGS. 4-6 with integration into a material upper 25 forming an underwear garment with a permeable layer 18, a filter 12, a nanomaterial cartridge 14, and an absorbent layer 16 at the crotch of the garment. Optionally the material upper 35 is a light flexible material.

In certain further implementations, the nanomaterial embedded device 10 is a sticker 40, shown in FIGS. 10-11, configured for placement in an undergarment such as underwear or alternatively on a conventional menstrual pad for analyte collection. In these implementations, the sticker 40 may include an adhesive edge 42, a mesh top layer 44, a nanomaterial cartridge/layer 14, and a removeable minimum fill area 46. That is, the sticker 40 includes an adhesive portion 42 for adhering the sticker 40 to the location of interest for collection. The mesh top layer 44 may be configured to trap cells of interest and allow smaller cells and DNA to pass through. The nanomaterial cartridge/layer 14 being configured to trap DNA/RNA of interest as described herein for further testing. The minimum fill area 46 configured to visually indicate to a user if enough fluid was been collected. In certain implementations the minimum fill area 46 is 15 mm³, although other volumes are possible and would be understood.

In certain implementations, the filter 12/mesh top layer 44 may be a commercially available filter 12. In some implementations the filter 12 is a commercial polymer filtration membrane. In some implementations the filter 12 may have a sensitivity over 90% and selectivity over 70%.

In certain implementations, the nanomaterial 14 is a silica sol gel, a templated silica sol gel, or a mesoporous silica xerogel with templated macropores for DNA binding configured to separate DNA/RNA from menstrual blood and/or vaginal discharge.

In various implementations, the nanomaterial 14 is formed from a silica precursor (such as TEOS or TMOS) with a catalyst (such as ammonia and/or hydrochloric acid), a picketing agent (such as Ludox-50), a micelle forming agent for pore templating (such as mesitylene and/or Pluronic-P123), and a support (such as PES). Various alternative or additional components for the nanomaterial 14 are possible.

In one example the nanomaterial 14 may be formed from TEOS as precursor used to create the gel, with colloidal silica (LUDOX TM-50) as an additive. Micelles may be used as templating agents. Pluronic P-123 may be used as a surfactant for templating. Various alternative components are possible.

In various implementations, a PES (polyethersulfone) membrane is used for precipitation of the nanomaterial 14. Optionally, the membrane has a 150 μm thickness and 8 um pore diameter.

As described above, the composite filter matrix/nanomaterial 14 is inserted into a tampon, pad, sticker, or other menstrual product. Templated macropores in the nanomaterial 14 and filter 12 allow for blood cells 30 and other cell types to pass through, larger cervical cells 32 may be trapped over the filtration membrane 12 and extracellular DNA 34 binds to the nanomaterial 14 (optionally mesoporous silica).

As would be appreciated, cervical cells 32 are >150 μm in diameter while blood cells 30 are 5-8 um in diameter allowing for separation of cells using appropriate pore sizes in the membrane 12 (i.e. 5-8 um diameter micropores) so blood 30 and unwanted particles pass through while trapping large cervical cells 32.

Securing DNA 34 to the separation matrix/nanomaterial 14 is needed for yielding high concentrations of DNA 34. Optionally, a mesoporous silica membrane with templated macropores using microemulsion is used for concentrating DNA 34/RNA in the nanomaterial cartridge 14.

In some implementations, a salt is used to facilitate the binding of negatively charged DNA 34 to the negatively charged silica surface in the nanomaterial cartridge 14. As would be appreciated silica binds to DNA under acidic conditions, and menstrual blood and lysing buffers are acidic. Silica is able to adsorb DNA with the help of a binding buffer and this binding can be reversed by the sure of a solvent, such as ethanol, as is described further herein.

The embedded device 10, such as a menstrual product or sticker, containing the sample may be dropped off, mailed, or otherwise delivered to a lab for processing. This processing may yield a pure nucleic acid sample that can be applied to hybridization assays and other forms of disease nucleic acid detection.

Optionally the embedded device 10 is worn by the patient/user for a time period, optionally a few hours, although other time periods are possible. Optionally the embedded device 10 is worn during a patient's menstrual cycle.

After use by the patient and sample collected it can be shipped, delivered, or dropped off at a lab/testing facility for further processing.

At the testing facility, the embedded device 10 may be processed and nanomaterial cartridge 14 and/or filter 12 removed from the absorbent layer 16, permeable layer 18, and the like. The cartridge 14 and/or filter 12 may then be exposed to a lysing agent to free DNA from cervical cells, removing anything other than DNA from the filter 12/cartridge. As would be understood, when exposed to chaotropic salts, the cell is lysed and the intracellular nucleic acid is freed from the cells and binds to the hierarchically porous silica. Unwanted cellular debris flows through the membrane and out of the cartridge 14.

In these implementations only nucleic acid found in cervical cells remains in the cartridge 14. This allows for elution via ethanol washing and yield of high purity HPV DNA. That is the cartridge 14 may then be exposed to ethanol to elute the DNA. This high cell DNA, and potentially high HPV, sample may be used for further testing such as and including HPV hybridization assays.

In one specific example, the nanomaterial embedded device 10 is received by a lab or other entity. The cartridge 14 is removed from other layers. A chaotropic solvent is added to the cartridge 14 to split open the cells of interest to release additional nucleic acid, which passively binds to the nanomaterial. Chaotropic solvents may include potassium chloride, sodium chloride, and the like as would be understood by those of skill in the art. The sample collected may then be centrifuged to eliminate any remaining blood and cell leftovers. An elution agent, such as ethanol, is then added to the nanomaterial cartridge, which may then be centrifuged to yield a nucleic acid sample. This sample can then be applied to hybridization assays and other nucleic acid detection tests without the need for PCR.

Although the disclosure has been described with references to various embodiments, persons skilled in the art will recognize that changes may be made in form and detail without departing from the spirit and scope of this disclosure.

What is claimed is:

1. A device for collection of vaginal discharge for medical testing comprising:
(a) a filter; and
(b) a nanomaterial in fluidic communication with the filter,
wherein the filter is configured to

2. The device of claim 1, wherein the vaginal discharge comprises menstrual blood.

3. The device of claim 1, further comprising an absorbent layer in fluidic communication with the nanomaterial.

4. The device of claim 1, wherein the device is a nanomaterial embedded tampon.

5. The device of claim 1, wherein the device is a nanomaterial embedded pad comprising at least a sticker layer.

6. The device of claim 1, wherein the device is a nanomaterial embedded underwear comprising at least a fabric upper.

7. The device of claim 1, wherein the device is a nanomaterial embedded sticker configured for placement in an undergarment.

8. The device of claim 1, wherein the nanomaterial is a silica sol gel with templated macropores.

9. The device of claim 1, wherein the filter is configured to trap cells of interest and allow other material to pass through while the nanomaterial is configured to bind DNA.

10. A device for collection of vaginal discharge or menstrual blood as an analyte of medical testing, comprising:
(a) a filter configured to separate cells of interest from other material; and
(b) a nanomaterial cartridge comprising a nanomaterial configured to passively adsorb DNA and segregate cells of different sizes.

11. The device of claim 10, wherein the cells of interest are cervical cells.

12. The device of claim 11, wherein the medical testing is HPV testing for cervical cancer diagnosis.

13. The device of claim 10, wherein the device is selected from a tampon, a pad, an underwear, and a sticker.

14. The device of claim 10, further comprising an absorbent layer.

15. The device of claim 10, wherein the nanomaterial is a silica sol gel with templated macropores.

16. The device of claim 10, further comprising a permeable layer.

17. The device of claim 10, wherein the nanomaterial cartridge may be removed from the device for medical testing.

18. A nanomaterial embedded sticker for collection of an analyte for medical testing, comprising:
(a) a mesh layer;
(b) a nanomaterial layer adjacent to the mesh layer;
(c) a minimum fill area within the nanomaterial configured to indicate a minimum fill for collection; and
(d) an adhesive portion at least encircling the nanomaterial layer.

19. The nanomaterial embedded sticker of claim 18, wherein the mesh layer is configured to trap one or more cells of interest and the nanomaterial layer is configured to adsorb DNA or RNA of interest.

20. The nanomaterial embedded sticker of claim 18, wherein the nanomaterial is a silica sol gel with templated macropores.
